(19) United States
(12) Patent Application Publication
(10) Pub. No.: US 2025/0042837 A1
(43) Pub. Date: Feb. 6, 2025

(54) A PROCESS FOR PURIFICATION OF CRUDE METHYL METHACRYLATE

(71) Applicant: MITSUBISHI CHEMICAL UK LIMITED, Billingham (GB)

(72) Inventors: Adam James Clarke, Wilton, Redcar (GB); Lars Anders Nicklas HJALMARSSON, Wilton, Redcar (GB)

(21) Appl. No.: 18/719,094

(22) PCT Filed: Dec. 12, 2022
(86) PCT No.: PCT/GB2022/053179

§ 371 Date: Jun. 12, 2024

(30) Foreign Application Priority Data
Dec. 13, 2021 (GB) 2118029.4

(51) Int. Cl. C07C 67/52; C07C 69/54

(52) U.S. Cl. CPC C07C 67/52; C07C 69/54

(57) ABSTRACT

A process for purifying a crude methyl methacrylate (MMA) stream is described, typically from one or more depolymerised (co)polymers comprising methyl methacrylate (MMA). The stream comprises MMA at a level of at least 80 wt % and ethyl acrylate (EA). The process includes the steps of:—
fractionally crystallising the said MMA stream to provide a fractionally crystallised MMA stream having a reduced EA content relative to the MMA stream immediately prior to fractional crystallisation.

The present invention relates to the process for purifying crude methyl methacrylate, typically from one or more depolymerised (co)polymers comprising methyl methacrylate (MMA). The impurities may comprise other monomers or depolymerisation by-products. The invention is directed to the removal of one such impurity in particular, that is, ethyl acrylate. Ethyl acrylate (EA) may be present as an impurity as a result of depolymerisation of copolymers containing ethyl acrylate residues and/or as a by-product of the depolymerisation process. Typically, it is present due to depolymerisation of copolymers of MMA and ethyl acrylate. It is known that ethyl acrylate is a “close boiler” to methyl methacrylate, i.e. that the boiling point of ethyl acrylate is close to that of methyl methacrylate. This makes it difficult to fully separate ethyl acrylate from methyl methacrylate using routine distillation. In particular, MMA is susceptible to polymerisation and distillation columns with large numbers of stages, high reflux ratios and high pressures are undesirable if this is to be avoided.

Alternative purification methods are known to the skilled person for the purification of products on an industrial scale. These include, but are not limited to specialised distillation techniques such as fractional distillation, reactive distillation, dividing wall distillation and spinning band distillation, reactive crystallisation, evaporative crystallisation, cooling crystallisation, evaporation, vapor compression evaporation, membrane filtration, reverse osmosis, ultrafiltration, gas-liquid chromatography, high pressure liquid chromatography (HPLC), gel permeation chromatography, ion exchange chromatography, adsorption, sublimation and liquid-liquid extraction, US20150119541, U.S. Pat. No. 6,380,427 and WO2020006058 all disclose various separation methods that may be used to remove target molecules from a product stream such as methacrylic acid. U.S. Ser. No. 10/808,262 discloses various methods for separating bioderived compounds from other components in the culture.

Furthermore, the purification of crude methyl methacrylate often requires the removal of multiple impurities depending on the source of the crude stream. It is known that EA can be separated from MMA by chromatography but such a process can be problematic on an industrial scale due to the associated clean up, downtime and running costs.

GB1235208 (Eastman Kodak) describes a process for the purification of alkyl methacrylates comprising fractional crystallisation to remove methyl butyrate which has a lower freezing point than MMA.

U.S. Pat. No. 6,670,501 B1 (Parten) discloses that close boilers with freezing points higher than methyl methacrylate can be separated from production process crude MMA by fractional crystallisation. Parten also illustrates that other production process impurities such as methyl isobutyrate (MiB) which actually has a lower freezing point can also be separated from methyl methacrylate.

Parten mentions lower MiB levels following fractional crystallisation. MiB has a freezing point of −85 C. However, the level of MiB in Parten in the crude stream and MMA crystals is only reduced to 56% of its original level from 2300 ppm to 1300 ppm.

Levels of EA in recycling streams would need to be reduced to much lower levels to be generally effective. Some copolymer sources for recycling streams may have a relatively high EA percentage and levels of 5 wt % and 10 wt % are not uncommon and even if lower levels such as 2500 ppm were present in some streams, a reduction to 1300 ppm would still not be satisfactory levels in a purified MMA monomer stream.

Surprisingly, it has nevertheless been found that of the available purification processes EA can be removed to a satisfactorily low level from an MMA crude stream by fractional crystallisation. The process to remove EA from MMA by fractional crystallisation is surprisingly more effective than the previously reported removal of MiB, despite EA having a more similar chemical structure to MMA compared to MiB, and EA having a “higher” freezing point (−71 C) that is closer to that of MMA (−48 C) compared to MiB (−85 C). Such similarities in structure may have been expected to result in co-crystallisation of EA with MMA and entrapment in the latter's crystal lattice and correspondingly low levels of removal of EA but the opposite effect has been found. The effect is particularly marked at already low levels of EA<2 wt %. It is surprising that even at these low levels the EA can be effectively removed to only a fraction of its former levels, typically, <100 ppm.

Furthermore, fractional crystallisation or a combination of fractional crystallisation with either pre- or post-fractional distillation purification steps provides a purified MMA monomer stream that is satisfactory under current REACH regulations. The purity levels of MMA according to the present invention are much higher and the level of EA very low by this technique.

According to the present invention there is provided a process for purifying crude MMA according to the claims.

Typically, the ratio of EA in the fractionally crystallised MMA stream compared to the crude MMA stream is <1:5, more typically, <1:10, most typically, <1:50. Advantageously, it has been found that in excess of 90% w/w of the EA can be removed from the crude MMA stream by the process of the invention, more typically, in excess of 95%, most typically, in excess of 97% w/w.

Advantageously, the MMA purity in the fractionally crystallised MMA stream may be in excess of 98 wt %, for example, in excess of 98.5 wt %, typically in excess of 99 wt % such as in excess of 99.5 wt %, 99.6 wt %, 99.7 wt %, 99.8 wt % or 99.9 wt %.

The process of the present invention may give yields (total final product/total feed) from the fractional crystallisation of: 80% such as >85%, >90% or >95%.

The fractional crystallisation process of the invention may use any form of fractional crystallisation known to the skilled person such as suspension crystallisation or layer crystallisation for example static crystallisation or falling film crystallisation.

A typical method of fractional crystallisation according to the invention includes a first stage comprising a first cooling phase of the crude stream to produce crystals of MMA and a residue liquor, an optional sweating phase to heat and partially remelt the crystals formed in the first cooling phase and produce sweated crystals and a sweating phase liquor, and a crystal melting phase to produce a purified liquid therefrom. The sweating phase is utilised to remove residual EA and other impurities from the impure portions of the crystals which melt at a lower temperature than the MMA. The sweating phase may include a single heating and remelting step or multiple heating and remelting steps such as 1, 2, 3, 4, or 5 such steps as required to effect the desired purity in the crystals. The residue liquor is removed after the cooling phase and sweating phase or after each heating and remelting step of the sweating phase. The residue liquors may be recycled to extract further MMA crystals after optionally mixing with further crude MMA feed streams.

Typically, at least one further crystallisation stage is performed that recrystallises the purified liquid stream produced from the first stage typically in accordance with the protocol of the first stage. Optionally, two or more further crystallisations of the liquid product are performed sequentially to produce progressively purer liquids. Up to 6 or 7 successive purification stages may be performed depending on the final product purity required. Advantageously, however, it has been found that satisfactory purification and EA removal may be achieved after 1 or 2 purification stages.

After the first cooling phase the residual liquor may be removed or recycled. Additionally, the liquor of the optional sweating phase may also be recycled for further crystallisations. This may improve the yield of the process.

Optionally, an initial nucleation step may take place where the temperature is temporarily lowered to initiate crystal formation and then raised to a higher temperature for slower crystal formation.

Accordingly, the cooling phase optionally includes an initial nucleation phase, where the temperature of the liquid to be purified is temporarily lowered to initiate crystal formation, and a crystal formation phase where the temperature is initially raised and optionally slowly lowered again for slower crystal formation during the rest of the cooling phase.

Typically, the liquid product stream to be purified is cooled to between about −45° C. and about −75° C. so that a part of the crude liquid product stream freezes to form crystals of solid methyl methacrylate and a residual liquor or supernatant, which is that part of the liquid product stream which remains unfrozen.

The level of impurities in the methyl methacrylate crystals may be affected by the rate at which the crude liquid product stream is cooled. The rate at which the liquid product stream is cooled may be controlled to optimise the separation of the methyl methacrylate from the impurities by minimising the amount of impurities contained in the crystals. A relatively slow rate of cooling has been found to produce methyl methacrylate crystals which contain a lower proportion of impurity than crystals formed as a result of faster cooling of the liquid product stream. The rate of cooling of the liquid product stream is preferably less than 30° C./min, more preferably less than 20° C./min and most preferably less than 10° C./min. An even lower rate of cooling such as less than 5 or 4 or 3 or 2 or 1 or 0.5 or 0.1° C./min may be used.

A suitable temperature range for crystal formation is from the saturation point of MMA in the liquor such as −48 to −70° C., more typically, −50 to −69° C., most typically, −52 to −69° C.

A suitable temperature for nucleation is below the freezing point of MMA such as in the range −53 to −75° C., more typically, −55 to −72° C., most typically, −58 to −62° C.

Therefore, a suitable protocol for the crystallisation is a nucleation cooling step in the range set out above until crystals begin to form, a heating step into the range for crystal formation as set out above which is above the nucleation temperature and slow cooling in the same temperature range.

Typically, the heating step will raise the temperature to −48 to −63° C. before optionally slow cooling in the temperature range from below −48 and down to −70° C.

Cooling effecting crystal formation may take place slowly so as to optimise crystal growth such as over a period of 1 to 20 hours, typically 4 to 10 hours, most typically 6 to 8 hours.

### Definitions

By (co)polymers herein is meant homo- or copolymers. The term copolymers includes polymers with two or more types of monomer residues and therefore includes terpolymers etc.

By crude MMA is meant any MMA that has impurities therein irrespective of whether some of the impurities have been removed. Accordingly, crude MMA includes MMA streams that have been purified prior to fractional crystallisation.

By Yield is meant the total final product/total feed. This Yield is based on the final product and this may have been subjected to 1 or >1 fractional crystallisation stages. Typically, 1 or 2 stages are sufficient, however 1-7 stages, more typically 1-3 stages of the purified product stream to yield the final product stream may be carried out.

The invention will now be described by way of example only with reference to the following examples and drawings in which:—

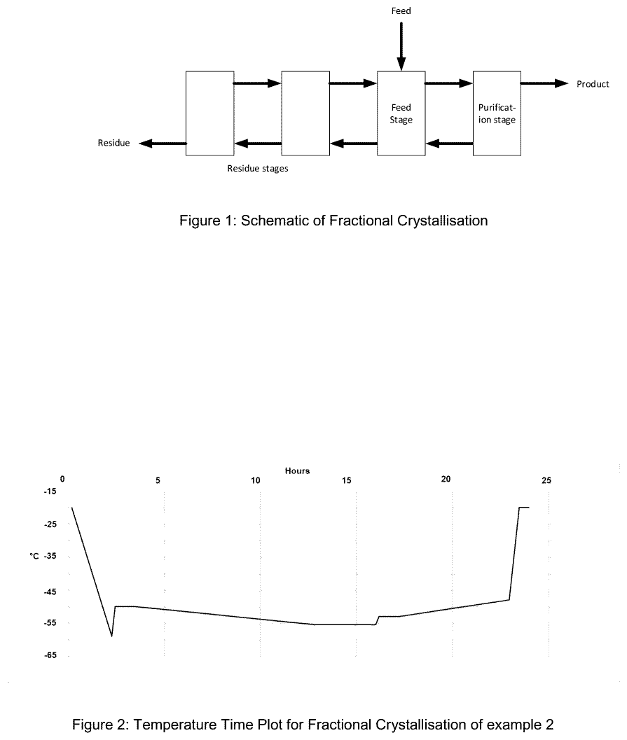

FIG. 1 is a schematic diagram of the fractional crystalliser;

FIG. 2 is a plot of temperature vs time for the fractional crystallisation of example 2.

### EXAMPLE 1

The results are based upon a synthetic crude with an MMA purity of approx. 94 wt % in addition to several impurities. The test showed that purification by crystallisation could dramatically reduce the EA concentration and provide an improved MMA purity even with several other impurities present. Two sequential fractional crystallisations were carried out.

The fractional crystallisation was a static crystallisation and it was performed on a crude mixture feed having the components detailed below in table 1.

TABLE 1

| | Melting | Feed | | Stage 1 | | Stage 2 | |
|---|---|---|---|---|---|---|---|
| Component | Point | Mixture | | Product | | Product | |
| Methyl | −48° C. | 94.92 | wt % | 99.36 | wt % | 99.90 | wt % |
| methacrylate | | | | | | | |
| (MMA) | | | | | | | |
| Ethyl | −71° C. | 0.17 | wt % | 155 | ppm | 10 | ppm |
| Acrylate | | | | | | | |
| (EA) | | | | | | | |
| Methyl | −85° C. | 0.76 | wt % | 0.12 | wt % | 206 | ppm |
| Isobutyrate | | | | | | | |
| (MiB) | | | | | | | |
| Methyl | −74° C. | 1.41 | wt % | 0.20 | wt % | 261 | ppm |
| Acrylate | | | | | | | |
| (MA) | | | | | | | |

As can be seen from the above results, the removal of EA is markedly higher in fractional crystallisation both at a single stage and a further stage crystallisation than other common impurities such as MiB and MA.

The product stream of the first stage was also recrystallised and recollected by melting in the 2^{nd} stage to further purify the product with similar beneficial EA removal.

In both stages, it was also possible to recycle the uncrystallised impure melt whether from the uncrystallised residue liquid or from the sweated crystals to crystallise at a later stage.

The general process is shown schematically in FIG. 1. In the feed stage, the impure feed liquid is cooled rapidly to a temperature below the freezing point of pure MMA such as −60° C. to effect nucleation of the MMA crystals and then heated to −48° C., the freezing point of MMA to begin slow crystallisation of the MMA crystals thereafter. Over several hours the liquor is gradually cooled to −65° C. to effect gradual crystallisation out of the mother liquor. Once a satisfactory amount of the mother liquor is crystallised, the process is stopped. In the purification stage, the remaining uncrystallised mother liquor is removed from the crystalliser and the crystals are partially remelted or ‘sweated’ by raising the temperature slowly until the necessary crystal purity is achieved. The remelt liquor is also then removed from the crystalliser and the remaining crystals of the necessary purity are fully melted, the purified liquor is then collected and analysed. The purified liquor of this first stage was then recrystallised in the 2^{nd} stage and the process above repeated. In table 1, only two stages are exemplified but multiple stages can be carried out as required.

Although not described, both the original mother liquor residue and the sweated crystal residue can be recycled and further crystallised to improve yield if necessary.

### EXAMPLE 2

A jacket vessel with an internal volume of 6 litres was used to perform fractional crystallisation on binary MMA-EA mixtures (5 wt % EA, 2 wt % EA and 1 wt % EA in MMA). These represent levels of EA that may be present in processed crude MMA. The crystallisation vessel was cooled using an external Unistat 705 refrigeration unit (Huber Offenburg/Germany) connected via insulated hoses. The system used a heat transfer fluid (Huber Thermal Fluid (HTF) DW-Therm M90.200.02). The crystalliser system temperature was controlled via the inbuilt temperature control functions of the Unistat 705 unit. Controlling the outlet HTF temperature in the range of 0 to −60° C. (FIG. 2).

The fractional crystallisation process consisted of nucleation, growth, multiple “sweating”, and melting phases of the final purified crystal product over a 23-hour period. As can be seen from FIG. 2, nucleation is effected by dropping the temperature relatively quickly to below the freezing point of MMA followed by relatively quick temperature increase to at or near the freezing point of MMA. The temperature is then slowly lowered to effect crystal growth as the freezing point falls in accordance with the purity of the supernatant liquor. The supernatant is then removed, and the temperature raised slightly towards the melting point of MMA to initiate sweating of the crystals. In FIG. 2, a single sweating step is shown. However, multiple sweating steps may be carried out. After each sweating phase, the liquid process fractions rich in EA content were removed from the crystallisation vessel to reduce the levels of this impurity in the crystallised MMA. The crystals were then melted by raising the temperature to −20 C to remove the purified MMA as a liquid. The separation efficiency was determined from the initial binary MMA-EA concentration and final melted product concentrations. Ethyl acrylate concentrations were determined by GC-FID analysis from two separate calibration curves, for levels 10-1 wt % EA in MMA and 500 to 5 ppmw levels EA in MMA.

The results are shown in table 2.

TABLE 2

| EA starting | EA product | Separation |
|---|---|---|
| concentration [wt %] | concentration [wt %] | efficiency [%] |
| 1.03 | 0.08 | 92.6 |
| 2.01 | 0.19 | 90.5 |
| 4.97 | 0.35 | 93.2 |

As can be seen form table 2, the separation efficiency is very high for removal of EA from the MMA product stream.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A process for purifying a crude methyl methacrylate (MMA) stream comprising MMA at a level of at least 80 wt % and ethyl acrylate (EA), comprising the steps of:—
(i) fractionally crystallising the said MMA stream to provide a fractionally crystallised MMA stream having a reduced EA content relative to the MMA stream immediately prior to fractional crystallisation.

2. The process according to claim 1, wherein one, two or more fractional crystallisations of the crude MMA stream are carried out in series to progressively remove the EA from the crude stream.

3. The process according to claim 1, wherein the crude stream is subject to a pre-fractional crystallisation purification step by a technique other than fractional crystallisation to produce a pre-fractional crystallisation purified crude stream having a MMA level of at least 92.5 wt %.

4. The process according to claim 3, wherein the crude MMA stream also comprises impurities other than EA and the process comprises the step of: —
a pre-fractional crystallisation purification by a technique other than fractional crystallisation of the crude MMA stream comprising the impurities other than EA and EA to obtain a pre-fractional crystallisation purified crude MMA stream comprising reduced content of the said impurities other than EA relative to the unpurified crude MMA stream and at least 92.5 wt % MMA prior to fractionally crystallising the said crude MMA stream in step (i).

5. The process according to claim 1, wherein the fractionally crystallised MMA stream contains impurities other than EA and the said stream is subjected to a post-fractional crystallisation purification step by a technique other than fractional crystallisation to provide reduced content of the said impurities other than EA relative to the fractionally crystallised stream content prior to said step.

6. The process according to claim 1, wherein the crude MMA stream comprises between 80-99 wt % MMA.

7. The process according to claim 1, wherein EA is present in the crude stream at a level of <10 wt %.

8. The process according to claim 1, wherein the EA is present in the crude stream at a level of from 0.01 to 10 wt %.

9. The process according to claim 1, wherein the crude MMA stream immediately prior to fractional crystallisation comprises at least 92.5 wt %.

10. The process according to claim 1, wherein the process produces a fractionally crystallised MMA stream with an MMA content of >98 wt %.

11. The process according to claim 1, wherein the fractionally crystallised MMA stream comprises at least 99 wt %.

12. The process according to claim 1, wherein the ratio of EA in the fractionally crystallised MMA stream compared to the crude MMA stream is <1:2.

13. The process according to claim 1, wherein the crude MMA stream is obtained from depolymerised (co)polymer(s) comprising MMA residues.

14. The process according to claim 13, wherein the crude MMA stream is obtained from depolymerised (co)polymer(s) comprising copolymers with MMA and EA residues.

15. The process according to claim 1, wherein the fractionally crystallised MMA stream comprises <5000 ppm EA.

16. The process according to claim 1, wherein the fractionally crystallised MMA stream comprises <350 ppm MiB.

17. The process according to claim 1, wherein the fractionally crystallised MMA stream comprises <500 ppm MA.

18. The process according to claim 1, wherein the fractional crystallisation process of step (i) is selected from suspension crystallisation or layer crystallisation.

19. The process according to claim 1, wherein one or more further fractional crystallisation step(s) is carried out on the product stream from step (i) and when carried out the levels and ratios of impurity and MMA following fractional crystallisation above may apply to the product stream of the said one or more further fractional crystallisations.

20. The process according to claim 1, wherein the fractional crystallisation includes a first stage comprising a first cooling phase of the crude stream to produce crystals and a residue, an optional sweating phase to heat and partially remelt the crystals formed in the first cooling phase and produce sweated crystals and a sweating phase liquor, and a crystal melting phase to produce a purified liquid therefrom.

21. The process according to claim 20, wherein at least one further crystallisation stage is performed that recrystallises the purified liquid stream produced from the first stage in accordance with the protocol of the first stage.

22. The process of claim 21, wherein two or more further such crystallisations are performed sequentially.

23. The process according to claim 20, wherein after the first cooling phase the residual liquor may be removed or recycled for further crystallisations.

24. The process according to claim 20, wherein the liquor of the sweating phase may be recycled for further crystallisations.

25. The process according to claim 20, wherein the cooling phase includes an initial nucleation phase, where the temperature of the stream to be purified is temporarily lowered to initiate crystal formation, and a crystal formation phase where the temperature is initially raised and optionally slowly lowered again for slower crystal formation during the rest of the cooling phase.

26. The process according to claim 1, wherein the fractional crystallisation includes a nucleation phase, typically, wherein the temperature of the stream to be purified is temporarily lowered to initiate crystal formation.

27. The process according to claim 1, wherein the fractional crystallisation includes a crystal growth phase.

28. The process according to claim 1, wherein the crystals formed during the fractional crystallisation are subjected to one or more sweating phases to heat and partially remelt the crystals formed.

29. The process according to claim 20, wherein the temperature range for crystal formation in the cooling phase of the crude MMA is −48 to −70° C.

30. The process according to claim 25, wherein the temperature for the nucleation phase is in the range −53 to −75° C.

31. The process according to claim 25, wherein the protocol for the fractional crystallisation comprises a nucleation phase cooling step, applied to the stream to be fractionally crystallised, in the range −53 to −75° C. until crystals begin to form.

32. The process according to claim 25, wherein the protocol for the fractional crystallisation comprises a heating step, applied to the stream to be fractionally crystallised, to the crystal formation phase initial temperature.

33. The process according to claim 32, wherein the initial temperature is above the nucleation temperature and less than or equal to −48° C.

34. The process according to claim 25, wherein the protocol for the crystallisation comprises an incremental cooling down of the stream from the initial temperature during the crystal formation phase and for the crystal formation period.

35. The process according to claim 34, wherein the crystal formation period is 1 to 20 hours.

36. The process according to claim 1, wherein the fractional crystallisation operating temperatures for crystal formation are between −45° C. and −70° C.

37. The process according to claim 1, wherein the fractional crystallisation operating pressure is between 0.1 and 5 bara.

38. The process according to claim 3, wherein the pre- and post-fractional crystallisation purification steps are independently selected from specialised distillation techniques comprising fractional distillation, reactive distillation, dividing wall distillation and spinning band distillation, reactive crystallisation, evaporative crystallisation, cooling crystallisation, evaporation, vapor compression evaporation, membrane filtration, reverse osmosis, ultrafiltration, gas-liquid chromatography, high pressure liquid chromatography (HPLC), gel permeation chromatography, ion exchange chromatography, adsorption, sublimation and liquid-liquid extraction.

39. The process according to claim 3, wherein one or more purification steps other than fractional crystallisation are carried out on the crude stream prior to fractional crystallisation of the crude stream.

40. The process according to claim 5, wherein one or more purification steps other than fractional crystallisation are carried out on the fractionally crystallised product stream.

41. An MMA stream produced by a process according to claim 1, wherein the MMA stream has a purity of at least 99 wt %, and comprises EA at <5000 ppm.

42. MMA produced by a process according to claim 1, wherein the MMA stream has a purity of at least 99 wt %, and comprises EA at <5000 ppm, typically, <1000 ppm EA, more typically <500 ppm EA, most typically, <100 ppm EA.

43. MMA having a purity of at least 99 wt %, and comprising EA at <5000 ppm.
